(19) United States
(12) Patent Application Publication
(10) Pub. No.: US 2020/0325226 A1
(43) Pub. Date: Oct. 15, 2020

(54) ANTI-PD-1 ANTIBODY FOR USE IN A METHOD OF TREATING A TUMOR

(71) Applicant: Bristol-Myers Squibb Company

(72) Inventors: Robin EDWARDS, Princeton, NJ (US); Han CHANG, Pennington, NJ (US); Michele CLEARY, Jamison, PA (US); Peter M. SZABO, Pennington, NJ (US); Joseph D. SZUSTAKOWSKI, Pennington, NJ (US); Patrik VITAZKA, Pennington, NJ (US)

(73) Assignee: Bristol-Myers Squibb Company, Princeton, NJ (US)

(21) Appl. No.: 16/306,380

(22) PCT Filed: Jun. 2, 2017
(86) PCT No.: PCT/US2017/035798

§ 371 Date: Nov. 30, 2018

(51) Int. Cl. C07K 16/28; G01N 33/574; C07K 16/30

(52) U.S. Cl. CPC C07K 16/2818; G01N 33/57423; A61K 2039/505; C07K 16/30; G01N 33/57484

(57) ABSTRACT

This disclosure provides a method for treating a subject afflicted with tumor, which method comprises administering to the subject an antibody or an antigen-binding portion thereof that specifically binds to a Programmed Death-1 (PD-1) receptor and inhibits PD-1 activity. In some embodiments, the tumor is derived from a non-small cell lung cancer (NSCLC). In some embodiments, the tumor expresses Programmed Death Ligand 1 (PD-L1), Serine/Threonine Kinase 11 (STK11), or both PD-L1 and STK11.

### FIELD OF THE INVENTION

This invention relates to methods for treating a tumor comprising administering to the subject an anti-Programmed Death-1 (PD-1) antibody, wherein the tumor expresses PD-L1 and/or wild-type STK11.

### BACKGROUND OF THE INVENTION

Human cancers harbor numerous genetic and epigenetic alterations, generating neoantigens potentially recognizable by the immune system (Sjoblom et al., (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down-regulate T cell activation and cytokine secretion upon binding to PD-1.

Nivolumab (formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of anti-tumor T-cell functions (U.S. Pat. No. 8,008,449; Wang et al., 2014 Cancer Immunol Res. 2(9):846-56).

NSCLC is the leading cause of cancer death in the U.S. and worldwide (NCCN GUIDELINES®, Version 3.2014—Non-Small Cell Lung Cancer, available at: www.nccn.org/professionals/physician_gls/pdf/nscl.pdf, last accessed May 14, 2014). NSCLCs are relatively insensitive to chemotherapy but patients with Stage IV disease who have a good performance status (PS) benefit from treatment with chemotherapeutic drugs, including platinum agents (e.g., cisplatin, carboplatin), taxane agents (e.g., paclitaxel, albumin-bound paclitaxel, docetaxel), vinorelbine, vinblastine, etoposide, pemetrexed, gemcitabine, and various combinations of these drugs.

### SUMMARY OF THE INVENTION

The present disclosure provides a method for treating a subject afflicted with a tumor comprising (i) determining an expression pattern of programmed death ligand 1 (PD-L1), and (ii) administering to the subject an antibody or an antigen-binding portion thereof that binds specifically to a Programmed Death-1 (PD-1) receptor and inhibits PD-1 activity (“anti-PD-1 antibody”) if the tumor exhibits a diffuse pattern of PD-L1 expression. In certain aspects, the disclosure provides methods for treating a subject afflicted with a tumor comprising (i) determining an expression pattern of PD-L1, and (ii) administering to the subject an anti-PD-1 antibody if the tumor exhibits a heterogeneous pattern of PD-L1 expression. In other aspects, the disclosure provides methods for treating a subject afflicted with a tumor comprising (i) determining an expression pattern of PD-L1, and (ii) administering to the subject an anti-PD-1 antibody if the tumor exhibits a tumor-stroma interface pattern of PD-L1 expression. In other aspects, the disclosure provides methods for identifying a subject afflicted with a tumor suitable for an anti-PD-1 antibody treatment comprising (i) determining an expression pattern of PD-L1, and (ii) administering to the subject an anti-PD-1 antibody if the tumor exhibits a diffuse pattern of PD-L1 expression. In still other aspects, the disclosure provides methods for identifying a subject afflicted with a tumor suitable for an anti-PD-1 antibody treatment comprising (i) determining an expression pattern of PD-L1, and (ii) administering to the subject an anti-PD-1 antibody if the tumor exhibits a heterogeneous pattern of PD-L1 expression. In certain embodiments, the methods disclosed herein further comprise identifying the patient as having a tumor that expresses STK11 prior to administration

In other aspects, the present disclosure relates to methods for treating a subject afflicted with a tumor comprising (i) identifying a subject having a STK11-positive tumor; and (ii) administering to the subject an anti-PD-1 antibody. In certain aspects, the disclosure relates to methods for treating a subject afflicted with a tumor comprising administering an anti-PD-1 antibody, wherein the patient is identified as having a STK11-positive tumor prior to the administration. In some aspects, the disclosure relates to methods for identifying a subject afflicted with a tumor suitable for an anti-PD-1 antibody treatment comprising (i) measuring an expression STK11 by the tumor, and (ii) administering to the subject an anti-PD-1 antibody if the tumor is STK11-positive. In some embodiments, the STK11 is wild-type STK11.

In some embodiments, the tumor is derived from a lung cancer. In some embodiments, the tumor is derived from a small cell lung cancer (SCLC) or a non-small cell lung cancer (NSCLC). In one embodiment, the tumor is derived from a NSCLC.

In some embodiments, the diffuse pattern of PD-L1 expression is characterized by a PD-L1 H-score of from about 60 to about 500, from about 80 to about 480, from about 100 to about 460, from about 120 to about 440, from about 140 to about 420, from about 160 to about 400, from about 180 to about 380, from about 200 to about 360, from about 200 to about 340, from about 200 to about 320, or from about 200 to about 300. In some embodiments, the diffuse pattern of PD-L1 expression is characterized by a PD-L1 H-score of at least about 200.

In some embodiments, the heterogeneous pattern of PD-L1 expression is characterized by a PD-L1 H-score of from about 1 to about 50, from about 5 to about 45, from about 10 to about 40, or from about 15 to about 35, and wherein the PD-L1 expression is restricted to one or more distinct portions of the tumor. In some embodiments, the heterogeneous pattern of PD-L1 expression is characterized by a PD-L1 H-score of at least about 15.

In some embodiments, the anti-PD-1 antibody cross-competes with nivolumab for binding to human PD-1. In some embodiments, the anti-PD-1 antibody binds to the same epitope as nivolumab. In some embodiments, the anti-PD-1 antibody is a chimeric, humanized, or human monoclonal antibody or a portion thereof. In some embodiments, the anti-PD-1 antibody is nivolumab.

In some embodiments, the anti-PD-1 antibody is administered at a dose ranging from at least about 0.1 mg/kg to at least about 10.0 mg/kg body weight once about every 1, 2 or 3 weeks. In one embodiment, the anti-PD-1 antibody is administered at a dose of at least about 3 mg/kg body weight once about every 2 weeks. In some embodiments, the anti-PD-1 antibody or antigen-binding portion thereof is administered at a flat dose about once every 1, 2, 3, or 4 weeks. In one embodiment, the anti-PD-1 antibody or antigen-binding portion thereof is administered at a flat dose or about 240 mg.

In other aspects, this disclosure provides a kit for treating a subject afflicted with a tumor, the kit comprising: (a) a dosage ranging from about 4 mg to about 500 mg of an anti-PD-1 antibody; and (b) instructions for using the anti-PD-1 antibody in any method described herein. In certain embodiments for treating human patients, the kit comprises an anti-human PD-1 antibody disclosed herein, e.g., nivolumab or pembrolizumab. In some embodiments, the kit further comprises an anti-PD-L1 antibody and/or an anti-STK11 antibody.

### EMBODIMENTS

E1. A method for treating a subject afflicted with a tumor comprising (i) determining an expression pattern of programmed death ligand 1 (PD-L1) and (ii) administering to the subject an antibody or an antigen-binding portion thereof that binds specifically to a Programmed Death-1 (PD-1) receptor and inhibits PD-1 activity (“anti-PD-1 antibody”) if the tumor exhibits a diffuse pattern of PD-L1 expression.

E2. A method for treating a subject afflicted with a tumor comprising (i) determining an expression pattern of PD-L1 and (ii) administering to the subject an anti-PD-1 antibody if the tumor exhibits a heterogeneous pattern of PD-L1 expression.

E3. A method for treating a subject afflicted with a tumor comprising (i) determining an expression pattern of PD-L1 and (ii) administering to the subject an anti-PD-1 antibody if the tumor exhibits a tumor-stroma interface pattern of PD-L1 expression.

E4. A method for identifying a subject afflicted with a tumor suitable for an anti-PD-1 antibody treatment comprising (i) determining an expression pattern of PD-L1 and (ii) administering to the subject an anti-PD-1 antibody if the tumor exhibits a diffuse pattern of PD-L1 expression.

E5. A method for identifying a subject afflicted with a tumor suitable for an anti-PD-1 antibody treatment comprising (i) determining an expression pattern of PD-L1 and (ii) administering to the subject an anti-PD-1 antibody if the tumor exhibits a heterogeneous pattern of PD-L1 expression.

E6. The method of any one of embodiments E1 to E5, further comprising identifying the patient as having a tumor that expresses STK11 prior to administration.

E7. A method for treating a subject afflicted with a tumor comprising (i) identifying a subject having a STK11-positive tumor; and (ii) administering to the subject an anti-PD-1 antibody.

E8. A method for treating a subject afflicted with a tumor comprising administering an anti-PD-1 antibody, wherein the patient is identified as having a STK11-positive tumor prior to the administration.

E9. A method for identifying a subject afflicted with a tumor suitable for an anti-PD-1 antibody treatment comprising (i) measuring an expression STK11 by the tumor and (ii) administering to the subject an anti-PD-1 antibody if the tumor is STK11-positive.

E10. The method of any one of embodiments E6 to E9, wherein the STK11 is wild-type STK11.

E11. The method of any one of embodiments E6 to E10, further comprising identifying the patient as having a tumor that expresses PD-L1 prior to administration.

E12. The method of any one of embodiments E1 to E11, wherein the tumor is derived from a lung cancer.

E13. The method of embodiment E12, wherein the tumor is derived from a small cell lung cancer (SCLC) or a non-small cell lung cancer (NSCLC).

E14. The method of embodiment E13, wherein the tumor is derived from an NSCLC.

E15. The method of anyone of embodiments E1 and E12 to E14, wherein the diffuse pattern of PD-L1 expression is characterized by a PD-L1 H-score of from about 60 to about 500, from about 80 to about 480, from about 100 to about 460, from about 120 to about 440, from about 140 to about 420, from about 160 to about 400, from about 180 to about 380, from about 200 to about 360, from about 200 to about 340, from about 200 to about 320, or from about 200 to about 300.

E16. The method of anyone of embodiments E1 and E12 to E15, wherein the diffuse pattern of PD-L1 expression is characterized by a PD-L1 H-score of at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 225, at least about 250, at least about 275, or at least about 300.

E17. The method of embodiment 15, wherein the diffuse pattern of PD-L1 expression is characterized by a PD-L1 H-score of at least about 200.

E18. The method of anyone of embodiments E1 and E12 to E14, wherein the heterogeneous pattern of PD-L1 expression is characterized by a PD-L1 H-score of from about 1 to about 50, from about 5 to about 45, from about 10 to about 40, or from about 15 to about 35, and wherein the PD-L1 expression is restricted to one or more distinct portions of the tumor.

E19. The method embodiment E18, wherein the heterogeneous pattern of PD-L1 expression is characterized by a PD-L1 H-score of at least about 5, at least about 10, at least about 15, at least about 20, at least about 25, at least about 30, at least about 35, or at least about 40.

E20. The method of embodiment E19, wherein the heterogeneous pattern of PD-L1 expression is characterized by a PD-L1 H-score of at least about 15.

E21. The method of any one of embodiments E1 to E5 and E12 to E20, wherein the expression pattern of PD-L1 is determined using an immunohistochemistry (IHC) assay.

E22. The method of embodiment E21, wherein the IHC assay is an automated IHC assay.

E23. The method of embodiment E21 or E22, wherein the IHC assay is performed using an anti-PD-L1 monoclonal antibody that specifically binds to the PD-L1 and wherein the anti-PD-L1 monoclonal antibody is selected from the group consisting of 28-8, 28-1, 28-12, 29-8, 5H1, and any combination thereof.

E24. The method of any one of embodiments E6 to E14, wherein the expression of STK11 is determined by detecting the presence of STK11 mRNA, the presence of STK11 protein, or both.

E25. The method of embodiment E24, wherein the presence of STK11 mRNA is determined using reverse transcriptase PCR.

E26. The method of embodiment E24, wherein the presence of STK11 protein is determined using an IHC assay.

E27. The method of embodiment E26, wherein the IHC assay is an automated IHC assay.

E28. The method of embodiment E26 or E27, wherein the IHC assay is performed using an anti-STK11 monoclonal antibody that specifically binds to the STK11.

E29. The method of any one of embodiments E1 to E6 and E11 to E23, wherein the tumor is characterized by having at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of tumor cells expressing PD-L1.

E30. The method of any one of embodiments E7 to E14, E24 to E28, and E30, wherein the STK11-positive tumor is characterized by having at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of tumor cells expressing STK11.

E31. The method of any one of embodiments E1 to E30, wherein the tumor exhibits high inflammation.

E32. The method of embodiment E31, wherein the inflammation is measured according to the expression of STK11.

E33. The method of any one of embodiments E1 to E32, wherein the anti-PD-1 antibody cross-competes with nivolumab for binding to human PD-1.

E34. The method of any one of embodiments E1 to E33, wherein the anti-PD-1 antibody binds to the same epitope as nivolumab.

E35. The method of any one of embodiments E1 to E34, wherein the anti-PD-1 antibody is a chimeric, humanized or human monoclonal antibody or a portion thereof.

E36. The method of any one of embodiments E1 to E35, wherein the anti-PD-1 antibody comprises a heavy chain constant region which is of a human IgG1 or IgG4 isotype.

E37. The method of any one of embodiments E1 to E36, wherein the anti-PD-1 antibody is nivolumab.

E38. The method of any one of embodiments E1 to E37, wherein the anti-PD-1 antibody is pembrolizumab.

E39. The method of any one of embodiments E1 to E38, wherein the anti-PD-1 antibody is administered at a dose ranging from at least about 0.1 mg/kg to at least about 10.0 mg/kg body weight once about every 1, 2 or 3 weeks.

E40. The method of embodiment E39, wherein the anti-PD-1 antibody is administered at a dose of at least about 3 mg/kg body weight once about every 2 weeks.

E41. The method of any one of embodiments E1 to E38, wherein the anti-PD-1 antibody or antigen-binding portion thereof is administered at a flat dose.

E42. The method of any one of embodiments E1 to E38 and E41, wherein the anti-PD-1 antibody or antigen-binding portion thereof is administered at a flat dose of at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500 or at least about 550 mg.

E43. The method of any one of embodiments E1 to E38, E41, and E42, wherein the anti-PD-1 antibody or antigen-binding portion thereof is administered at a flat dose or about 240 mg.

E44. The method of any one of embodiments E1 to E38, E41 and E42, wherein the anti-PD-1 antibody or antigen-binding portion thereof is administered at a flat dose about once every 1, 2, 3 or 4 weeks.

E45. The method of any one of embodiments E1 to E44, wherein the anti-PD-1 antibody is administered for as long as clinical benefit is observed or until unmanageable toxicity or disease progression occurs.

E46. The method of any one of embodiments E1 to E45, wherein the anti-PD-1 antibody is formulated for intravenous administration.

E47. The method of any one of embodiments E1 to E46, wherein the anti-PD-1 antibody is administered at a subtherapeutic dose.

E48. The method of any one of embodiments E1 to E47, wherein the administering treats the tumor.

E49. The method of any one of embodiments E1 to E48, wherein the administering reduces the size of the tumor.

E50. The method of embodiment E49, wherein the size of the tumor is reduced by at least about 10%, about 20%, about 30%, about 40%, or about 50% compared to the tumor size prior to the administration.

E51. The method of any one of embodiments E1 to E50, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

E52. The method of any one of embodiments E1 to E51, wherein the subject exhibits stable disease after the administration.

E53. The method of any one of embodiments E1 to E51, wherein the subject exhibits a partial response after the administration.

E54. The method of any one of embodiments E1 to E51, wherein the subject exhibits a complete response after the administration.

E55. A kit for treating a subject afflicted with a tumor, the kit comprising:
(a) a dosage ranging from about 4 mg to about 500 mg of an anti-PD-1 antibody; and
(b) instructions for using the anti-PD-1 antibody in the method of any of embodiments E1 to E54.

E56. The kit of embodiment E55, further comprising an anti-PD-L1 antibody.

E57. The kit of embodiment E55 or E56, further comprising an anti-STK11 antibody.

### BRIEF DESCRIPTION OF THE DRAWINGS

FIGS. 1A-1D present four immunohistochemistry (IHC) images showing distinct patterns of PD-L1 expression in NSCLC commercial tumors. The patterns of PD-L1 expression are designated diffuse (FIG. 1A), heterogeneous (FIG. 1B), tumor-stroma interface (FIG. 1C), and negative (FIG. 1D).

FIGS. 2A and 2B show the distribution of PD-L1 H-scores in each PD-L pattern as shown in FIGS. 1A-D, i.e., diffuse (D), heterogeneous (H), negative (N), and tumor-stroma interface (T) (FIG. 2A), and the distribution of PD-L1 H-scores in two NSCLC subtypes, namely adenocarcinoma and squamous cell carcinoma (FIG. 2B).

FIGS. 3A-3C show IHC images corresponding to diffuse (FIG. 3A), tumor-stroma interface (FIG. 3B), and negative (FIG. 3C) PD-L expression patterns corresponding to trial biopsies from patients treated with nivolumab monotherapy.

FIG. 4 shows the PD-L H-score of patients, categorized by tumor grade, undergoing nivolumab monotherapy. The predominant PD-L pattern in the majority of complete responders (CRs) and partial responders (PRs) is the diffuse pattern. TS-IF=tumor-stroma interface, SD=stable disease, PD=progressive disease, BOR=best overall response.

FIGS. 5A and 5B show the Overall CI Score (FIG. 5A) and PD-L1 CI Score (FIG. 5B) according to the PD-L tumor predominant pattern. TS-IF=tumor-stroma interface.

FIG. 6 presents a multiplexed IHC image stained for PD-L1, CD68, and CD3.

FIGS. 7A and 7B show PD-L1 expression in NSCLC tumors according to the PD-L1 expression pattern as measured using RNA sequencing (FIG. 7A), and the mutation load in NSCLC tumors according to the PD-L expression pattern as measured using exome sequencing (FIG. 7B).

FIGS. 8A and 8B show the relationship between the number of missense mutations in NSCLC tumors versus overall inflammation as measured by the CI Score (FIG. 8A), and the relationship between the number of missense mutation in NSCLC tumors versus PD-L1+ inflammation as measured by the PDLPipos CI Score (FIG. 8B).

FIGS. 9A and 9B show the frequency of mutations in different biomarkers (TP53, STK11, KEAP1, KRAS, EGFR, and MET) versus the observed PD-L expression pattern in FIG. 9A. D=diffuse, H=heterogenous, I=tumor-stroma interface, N=negative. FIG. 9B shows PD-L1 expression measured by RNA sequencing (RNAseq) versus presence (“y”) or absence (“n”) of STK11 mutations.

FIGS. 10A-10C show the relationship between presence (“y”) or absence (“n”) of STK11 mutations and PD-L1+ CI Score (FIG. 10A). The numeric data corresponding to the information presented in FIG. 10A is shown in FIG. 10B. FIG. 10C shows numeric data corresponding to overall inflammation scores in NSCLC tumors depending on the presence (“STK11-MUT”) or absence (“STK11-WT”) of STK11 mutations.

FIG. 11 shows an immunoprint analysis of twenty-four (24) NSCLC tumor samples in which levels of FOLR2, VSIG4, CD163, CLEC4D, CSF1R, CD86, MS4A1, CD79B, CD19, KIR2DS4, KIR2DL4, CD3E, CCR4, CCR8, and CD8A were analyzed to classify the samples according to inflammation patterns (sigClass). Samples were classified as low (“sigClass low”), medium (“sigClass med”), or high (“sigClass hi”) inflammation. Samples were classified also according to presence (“STK11 mut”) or absence (“STK11 wt”) of STK11 mutations. In addition, samples were classified according to PD-L1 expression pattern as negative (“PDL1_pattern 2 Negative”), diffuse (“PDL1_pattern 2 Diffuse”), heterogeneous (“PDL1_pattern 2 Heterogeneous”), or tumor-stroma interface (“PDL1_pattern 2 TS”).

### DETAILED DESCRIPTION OF THE INVENTION

The present invention relates to methods for treating a subject afflicted with a tumor comprising (i) determining an expression pattern of programmed death ligand 1 (PD-L1), and (ii) administering to the subject an antibody or an antigen-binding portion thereof that binds specifically to a Programmed Death-1 (PD-1) receptor and inhibits PD-1 activity (“anti-PD-1 antibody”) if the tumor exhibits a diffuse pattern of PD-L1 expression. In other aspects, the present invention relates to methods for treating a subject afflicted with a tumor comprising (i) identifying a subject having a STK11-positive tumor, and (ii) administering to the subject an anti-PD-1 antibody. In certain embodiments, the tumor is derived from a NSCLC.

### Terms

In order that the present disclosure can be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Routes of administration for the anti-PD-1 antibody include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the combination is administered via a non-parenteral route, in some embodiments, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG, and IgM. IgG subclasses are also well known to those in the art and include but are not limited to human IgG1, IgG2, IgG3, and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 can, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic, or other techniques known to those skilled in the art.

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDRs of a non-human antibody are replaced with corresponding amino acids derived from human immunoglobulins. In one embodiment of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-PD-1 antibody binds specifically to PD-1.

An “antigen-binding portion” of an antibody (also called an “antigen-binding fragment”) refers to one or more fragments of an antibody that retain the ability to bind specifically to the antigen bound by the whole antibody.

A “cancer” refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream. In some embodiments, the cancer is any cancer disclosed herein. In embodiments, the cancer is lung cancer. In certain embodiments, the lung cancer is non-small cell lung cancer (NSCLC). In embodiments, the NSCLC has a squamous histology (squamous NSCLC). In other embodiments, the NSCLC has a non-squamous histology (non-squamous NSCLC). A “cancer” can include a tumor. A “tumor” refers to all neoplastic cell growth and proliferation, whether malignant or benign, and all pre-cancerous and cancerous cells and tissues.

“Serine/Threonine Kinase 11” or “STK11” (also known as “Polarization-Related Protein LKB1,” “Renal Carcinoma Antigen NY-REN-19,” “Liver Kinase B1,” “EC 2.7.11.1,” and “HLKB1”) refers to a member of the serine/threonine kinase family that regulates cell polarity and functions as a tumor suppressor. STK11 controls the activity of AMP-activated protein kinase (AMPK) family members, thereby playing a role in various processes such as cell metabolism, cell polarity, apoptosis, and DNA damage response. STK11 is ubiquitously expressed, with the strongest expression in the testis and the fetal liver. STK11 is commonly inactivated in NSCLC, especially in tumors harboring KRAS mutations. As described herein, mutated STK11, e.g., loss of wild type expression of STK11, correlates with decreased or aberrant PD-L1 expression in tumors derived from an SCLC. In some embodiments, mutated STK11, e.g., loss of wild type expression of STK11, occurs in a tumor derived from an SCLC, wherein the tumor either expresses or does not express wild type KRAS (e.g., the tumor has or does not have a KRAS mutation). In some embodiments, the STK11 mutant is an STK11 mutant previously described in, e.g., Koyama et al., Cancer Res. 76(5):999-1008 (2016), and/or Skoulidis et al., Cancer Discov. 5(8):860-77 (2015), both of which are incorporated by reference herein in their entirety.

The term “immunotherapy” refers to the treatment of a subject afflicted with, at risk of contracting, or suffering a recurrence of a disease by a method comprising inducing, enhancing, suppressing, or otherwise modifying an immune response. “Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease.

“PD-L1 positive” as used herein can be interchangeably used with “PD-L1 expression of at least about 1%.” In one embodiment, the PD-L1 expression can be used by any methods known in the art. In another embodiment, the PD-L1 expression is measured by an automated IHC. PD-L1 positive tumors can thus have at least about 1%, at least about 2%, at least about 5%, at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100% of the tumor cells expressing PD-L1 as measured by an automated IHC. In certain embodiments, “PD-L1 positive” means that there are at least 100 cells that express PD-L1 on the surface of the cells.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “subtherapeutic dose” means a dose of a therapeutic compound (e.g., an antibody) that is lower than the usual or typical dose of the therapeutic compound when administered alone for the treatment of a hyperproliferative disease (e.g., cancer).

By way of example, an “anti-cancer agent” promotes cancer regression in a subject or prevents further tumor growth. In certain embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ, and/or organism level (adverse effects) resulting from administration of the drug.

By way of example for the treatment of tumors, a therapeutically effective amount of an anti-cancer agent can inhibit cell growth or tumor growth by at least about 10%, by at least about 20%, by at least about 40%, by at least about 60%, by at least about 70%, by at least about 80%, by at least about 90%, at least about 95%, or at least about 100% relative to untreated subjects. In other embodiments of the invention, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns”.

An “immune-related response pattern” refers to a clinical response pattern often observed in cancer patients treated with immunotherapeutic agents that produce anti-tumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease. A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In certain embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., 10% or 20%). For example, about 3 mg can include any number between 2.7 mg and 3.3 mg (for 10% or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days one day, i.e., every six days to every eight days. “Once about every two weeks” can include every fourteen days three days, i.e., every eleven days to every seventeen days. Similar approximations apply, for example, to once about every three weeks, once about every four weeks, once about every five weeks, once about every six weeks and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The term “weight-based dose” as referred to herein means that a dose administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

The use of the term “fixed dose” with regard to a method of the invention means that two or more different antibodies in a single composition (e.g., anti-PD-1 antibody and a second antibody) are present in the composition in particular (fixed) ratios with each other. In some embodiments, the fixed dose is based on the weight (e.g., mg) of the antibodies. In certain embodiments, the fixed dose is based on the concentration (e.g., mg/ml) of the antibodies. In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first antibody (e.g., anti-PD-1 antibody) to mg second antibody. For example, the 3:1 ratio of an anti-PD-1 antibody and a second antibody can mean that a vial can contain about 240 mg of the anti-PD-1 antibody and 80 mg of the second antibody or about 3 mg/ml of the anti-PD-1 antibody and 1 mg/ml of the second antibody.

The use of the term “flat dose” with regard to the methods and dosages of the invention means a dose that is administered to a patient without regard for the weight or body surface area (BSA) of the patient. The flat dose is therefore not provided as a mg/kg dose, but rather as an absolute amount of the agent (e.g., the anti-PD-1 antibody). For example, a 60 kg person and a 100 kg person would receive the same dose of an antibody (e.g., 240 mg of an anti-PD-1 antibody).

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### Methods of the Invention

This disclosure provides a method for treating a subject afflicted with a tumor comprising (i) determining an expression pattern of Programmed Death Ligand 1 (PD-L1), and (ii) administering to the subject an antibody or an antigen-binding portion thereof that binds specifically to a Programmed Death-1 (PD-1) receptor and inhibits PD-1 activity (“anti-PD-1 antibody”) if the tumor exhibits a diffuse pattern of PD-L1 expression. In certain aspects, the disclosure provides methods for treating a subject afflicted with a tumor comprising (i) determining an expression pattern of PD-L1, and (ii) administering to the subject an anti-PD-1 antibody if the tumor exhibits a heterogeneous pattern of PD-L1 expression. In other aspects, the disclosure provides methods for treating a subject afflicted with a tumor comprising (i) determining an expression pattern of PD-L1, and (ii) administering to the subject an anti-PD-1 antibody if the tumor exhibits a tumor-stroma interface pattern of PD-L1 expression. In other aspects, the disclosure provides methods for identifying a subject afflicted with a tumor suitable for an anti-PD-1 antibody treatment comprising (i) determining an expression pattern of PD-L1, and (ii) administering to the subject an anti-PD-1 antibody if the tumor exhibits a diffuse pattern of PD-L1 expression. In still other aspects, the disclosure provides methods for identifying a subject afflicted with a tumor suitable for an anti-PD-1 antibody treatment comprising (i) determining an expression pattern of PD-L1, and (ii) administering to the subject an anti-PD-1 antibody if the tumor exhibits a heterogeneous pattern of PD-L1 expression. In certain embodiments, the methods disclosed herein further comprise identifying the patient as having a tumor that expresses STK11 prior to administration.

In other aspects, the present disclosure relates to methods for treating a subject afflicted with a tumor comprising (i) identifying a subject having a STK11-positive tumor (e.g., STK11 wild-type), and (ii) administering to the subject an anti-PD-1 antibody. In certain aspects, the disclosure relates to methods for treating a subject afflicted with a tumor comprising administering an anti-PD-1 antibody, wherein the patient is identified as having a STK11-positive tumor prior to the administration. In some aspects, the disclosure relates to methods for identifying a subject afflicted with a tumor suitable for an anti-PD-1 antibody treatment comprising (i) measuring an expression STK11 by the tumor, and (ii) administering to the subject an anti-PD-1 antibody if the tumor is STK11-positive. In some embodiments, the STK11 is wild-type STK11.

In other aspects, the present disclosure relates to methods for treating a subject afflicted with a tumor comprising (i) identifying a subject having a STK11-negative tumor; and (ii) not administering to the subject an anti-PD-1 antibody or terminating or augmenting an anti-PD-1 antibody therapy. Other aspects of the present disclosure relate to methods of identifying a subject afflicted with a tumor that is not suitable for an anti-PD-1 antibody treatment comprising (i) measuring an expression STK11 by the tumor, and (ii) not administering to the subject an anti-PD-1 antibody or terminating or augmenting an anti-PD-1 antibody therapy if the tumor is STK11-negative or if the tumor expresses an inactive STK11 mutant.

In certain embodiments, the tumor is derived from a NSCLC. In some embodiments, the subject is a human patient. In certain embodiments, the subject is a chemotherapy-naïve patient (e.g., a patient who has not previously received any chemotherapy). In other embodiments, the subject for the present combination therapy has received another cancer therapy (e.g., a chemotherapy), but is resistant or refractory to such another cancer therapy. In certain specific embodiments, the subject for the present therapy has tumor cells expressing mutated forms of the EGFR, KRAS, and/or STK11 gene. In some embodiments, the subject for the present therapy has tumor cells that express both wild type STK11 and mutated STK11. In other embodiments, the subject for the present therapy has tumor cells that express only the wild type form of STK11. In some embodiments, the tumor expresses one or more genes selected from TP53, KEAP1, KRAS, EGFR, MET, and one or more mutant variants thereof. In some embodiments, the tumor expresses STK11 and one or more genes selected from TP53, KEAP1, KRAS, EGFR, MET, and one or more mutant variants thereof.

In certain embodiments, the subject has tumor cells that are PD-L1 positive (PD-L1+). In certain embodiments, the subject has cancer cells that are PD-L1 negative (PD-L1−). In some embodiments, the subject never smoked. In certain embodiments, the subject formerly smoked. In one embodiment, the subject currently smokes. In certain embodiments, the subject has cancer cells that are squamous. In certain embodiments, the subject has cancer cells that are non-squamous.

In some embodiments, the diffuse pattern of PD-L1 expression is characterized by a PD-L1 H-score of from about 60 to about 500, from about 70 to about 490, from about 80 to about 480, from about 90 to about 470, from about 100 to about 460, from about 110 to about 450, from about 120 to about 440, from about 130 to about 430, from about 140 to about 420, from about 150 to about 410, from about 160 to about 400, from about 170 to about 390, from about 180 to about 380, from about 190 to about 370, from about 200 to about 360, from about 20 to about 350, from about 200 to about 340, from about 200 to about 330, from about 200 to about 320, from about 200 to about 310, or from about 200 to about 300. In certain embodiments, the diffuse pattern of PD-L1 expression is characterized by a PD-L1 H-score of at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 225, at least about 230, at least about 240, at least about 250, at least about 260, at least about 270, at least about 275, at least about 280, at least about 290, or at least about 300. In certain embodiments, the diffuse pattern of PD-L1 expression is characterized by a PD-L1 H-score of at least about 200. In other embodiments, the diffuse pattern of PD-L1 expression is characterized by a PD-L1 H-score of at least about 240. In certain embodiments, the diffuse pattern of PD-L1 expression is characterized by a PD-L1 H-score of at least about 260.

In some embodiments, the heterogeneous pattern of PD-L1 expression is characterized by a PD-L1 H-score of from about 1 to about 50, from about 5 to about 45, from about 10 to about 40, or from about 15 to about 35, and wherein the PD-L1 expression is restricted to one or more distinct portions of the tumor. In some embodiments, the heterogeneous pattern of PD-L1 expression is characterized by a PD-L1 H-score of at least about 5, at least about 10, at least about 15, at least about 20, at least about 25, at least about 30, at least about 35, or at least about 40. In one embodiment, the heterogeneous pattern of PD-L1 expression is characterized by a PD-L1 H-score of at least about 15. In another embodiment, the heterogeneous pattern of PD-L1 expression is characterized by a PD-L1 H-score of at least about 20. In some embodiments, the heterogeneous pattern of PD-L1 expression is characterized by a portion of the tumor comprising at least 10, at least 20, at least 30, at least 40, at least 50, at least 60, at least 70, at least 80, at least 90, or 100, at least 120, or at least 150 expressing PD-L1. In certain embodiments, at least about 30%, at least about 40%, at least about 50%, at least about 60%, at least about 70%, at least about 80%, at least about 90%, at least about 95%, or about 100% of the cells within the portion of the tumor express PD-L1.

In some embodiments, the tumor-stroma interface PD-L1 expression is characterized by expression of PD-L1 by tumor cells adjacent (e.g., with in about 1 cell diameter, about 2 cell diameters, about 3 cell diameters, about 4 cell diameters, about 5 cell diameters, about 6 cell diameters, about 7 cell diameters, about 8 cell diameters, about 9 cell diameters, or about 10 cell diameters) of the stroma. In certain embodiments, the tumor-stroma interface PD-L1 expression is characterized by PD-L1 expression on the surface of the tumor.

In certain embodiments, the therapy of the present invention (e.g., administration of an anti-PD-1 antibody) effectively increases the duration of survival of the subject. In some embodiments, the anti-PD-1 antibody therapy of the present invention increases the progression-free survival of the subject. In certain embodiments, the anti-PD-1 antibody therapy of the present invention increases the progression-free survival of the subject in comparison to standard-of-care therapies. After the administration of an anti-PD-1 antibody therapy, the subject having a tumor can exhibit an overall survival of at least about 10 months, at least about 11 months, at least about 12 months, at least about 13 months, at least about 14 months at least about 15 months, at least about 16 months, at least about 17 months, at least about 18 months, at least about 19 months, at least about 20 months, at least about 21 months, at least about 22 months, at least about 23 months, at least about 2 years, at least about 3 years, at least about 4 years, or at least about 5 years after the administration.

In other embodiments, the duration of survival or the overall survival of the subject is increased by at least about 1 month, at least about 2 months, at least about 3 months, at least about 4 months, at least about 6 months, or at least about 1 year when compared to another subject treated with only a standard-of-care therapy (e.g., docetaxel) or a different dosing schedule of the therapy. For example, the duration of survival or the overall survival of the subject treated with an anti-PD-1 antibody disclosed herein is increased by at least about 5%, at least about 10%, at least about 15%, at least about 20%, at least about 25%, at least about 30%, at least about 40%, at least about 50%, or at least about 75% when compared to another subject treated with only a standard-of-care therapy (e.g., docetaxel) or a different dosing schedule of the combination therapy.

In certain embodiments, the therapy of the present invention effectively increases the duration of progression free survival of the subject. In some embodiments, the subject exhibits a progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years.

The PD-L1 or STK11 status of a tumor in a subject can be measured prior to administering any composition or utilizing any method disclosed herein. In one embodiment, the PD-L1 or STK11 expression level of a tumor is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100%. In another embodiment, the PD-L1 or STK11 status of a tumor is at least about 1%. In other embodiments, the PD-L1 or STK11 status of the subject is at least about 5%. In a certain embodiment, the PD-L1 or STK11 status of a tumor is at least about 10%. In a one embodiment, the PD-L1 or STK11 status of the tumor is at least about 25%. In a particular embodiment, the PD-L1 status of the tumor is at least about 50%.

In certain embodiments, the tumor can exhibit a high level of inflammation. Increased inflammation can be indicative of a diffuse PD-L1 expression pattern. Accordingly, high tumor inflammation can be indicative of responsiveness to an anti-PD-1 antibody therapy. In certain embodiments, the inflammation can be measured according to the expression of STK11, PD-L1, TP53, KEAP1, KRAS, EGFR, and/or MET.

In some embodiments, the median progression-free survival of a subject with a tumor that has ≥1% PD-L1 expression is at least about 1 week, at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year longer than the median progression-free survival of a subject with a tumor with a <1% PD-L1 expression. In some embodiments, the progression-free survival of a subject with a tumor that has ≥1% PD-L1 expression is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about eighteen months, at least about 2 years, at least about 3 years, at least about 4 years, or at least about 5 years.

In some embodiments, the administering of the anti-PD-1 antibody treats the tumor. In certain embodiments, the administering reduces the size of the tumor. In one embodiment, the size of the tumor is reduced by at least about 10%, about 20%, about 30%, about 40%, or about 50% compared to the tumor size prior to the administration. In other embodiments, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration. In some embodiments, the subject exhibits stable disease after the administration. In some embodiments, the subject exhibits a partial response after the administration. In some embodiments, the subject exhibits a complete response after the administration. In some embodiments, the subject exhibits an improved objective response rate (ORR) after the administration as compared to a subject treated with a standard of care treatment.

In order to assess the PD-L1 expression and/or the STK11 expression, in one embodiment, a test tissue sample can be obtained from the patient who is in need of the therapy. In another embodiment, the assessment of PD-L1 and/or STK11 expression can be achieved without obtaining a test tissue sample. In some embodiments, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express PD-L1 on the surface of the cells based on an assessment that the proportion of cells in the test tissue sample that express PD-L1 on the cell surface is higher than a predetermined threshold level.

In any of the methods comprising the measurement of PD-L1 and/or STK11 expression in a test tissue sample, however, it should be understood that the step comprising the provision of a test tissue sample obtained from a patient is an optional step. It should also be understood that in certain embodiments the “measuring” or “assessing” step to identify, or determine the number or proportion of, cells in the test tissue sample that express PD-L1 and/or STK11 (e.g., the expression of PD-L1 on the cell surface) is performed by a transformative method of assaying for PD-L1 and/or STK11 expression, for example by performing a reverse transcriptase-polymerase chain reaction (RT-PCR) assay or an IHC assay. In certain other embodiments, no transformative step is involved and PD-L1 and/or STK11 expression is assessed by, for example, reviewing a report of test results from a laboratory. In certain embodiments, the steps of the methods up to, and including, assessing PD-L1 and/or STK11 expression provides an intermediate result that may be provided to a physician or other healthcare provider for use in selecting a suitable candidate for the anti-PD-1 antibody or anti-PD-L1 antibody therapy. In certain embodiments, the steps that provide the intermediate result is performed by a medical practitioner or someone acting under the direction of a medical practitioner. In other embodiments, these steps are performed by an independent laboratory or by an independent person such as a laboratory technician.

In certain embodiments of any of the present methods, the proportion of cells that express PD-L1 and/or STK11 is assessed by performing an assay to determine the presence of PD-L1 and/or STK11 RNA. In further embodiments, the presence of PD-L1 and/or STK11 RNA is determined by RT-PCR, in situ hybridization or RNase protection. In other embodiments, the proportion of cells that express PD-L1 and/or STK11 is assessed by performing an assay to determine the presence of PD-L1 and/or STK11 polypeptide. In further embodiments, the presence of PD-L1 and/or STK11 polypeptide is determined by immunohistochemistry (IHC), enzyme-linked immunosorbent assay (ELISA), in vivo imaging, or flow cytometry. In some embodiments, PD-L1 and/or STK11 expression is assayed by IHC. In other embodiments of all of these methods, cell surface expression of PD-L1 and/or STK11 is assayed using, e.g., IHC or in vivo imaging.

Imaging techniques have provided important tools in cancer research and treatment. Recent developments in molecular imaging systems, including positron emission tomography (PET), single-photon emission computed tomography (SPECT), fluorescence reflectance imaging (FRI), fluorescence-mediated tomography (FMT), bioluminescence imaging (BLI), laser-scanning confocal microscopy (LSCM), and multiphoton microscopy (MPM) will likely herald even greater use of these techniques in cancer research. Some of these molecular imaging systems allow clinicians to not only see where a tumor is located in the body, but also to visualize the expression and activity of specific molecules, cells, and biological processes that influence tumor behavior and/or responsiveness to therapeutic drugs (Condeelis and Weissleder, “In vivo imaging in cancer,” Cold Spring Harb. Perspect. Biol. 2(12):a003848 (2010)). Antibody specificity, coupled with the sensitivity and resolution of PET, makes immunoPET imaging particularly attractive for monitoring and assaying expression of antigens in tissue samples (McCabe and Wu, “Positive progress in immunoPET—not just a coincidence,” Cancer Biother. Radiopharm. 25(3):253-61 (2010); Olafsen et al., “ImmunoPET imaging of B-cell lymphoma using 124I-anti-CD20 scFv dimers (diabodies),” Protein Eng. Des. Sel. 23(4):243-9 (2010)). In certain embodiments of any of the present methods, PD-L1 and/or STK11 expression is assayed by immunoPET imaging. In certain embodiments of any of the present methods, the proportion of cells in a test tissue sample that express PD-L1 and/or STK11 is assessed by performing an assay to determine the presence of PD-L1 and/or STK11 polypeptide on the surface of cells in the test tissue sample. In certain embodiments, the test tissue sample is a FFPE tissue sample. In other embodiments, the presence of PD-L1 and/or STK11 polypeptide is determined by IHC assay. In further embodiments, the IHC assay is performed using an automated process. In some embodiments, the IHC assay is performed using an anti-PD-L1 monoclonal antibody to bind to the PD-L1 polypeptide. In other embodiments, the IHC assay is performed using an anti-STK11 monoclonal antibody to bind to the STK11 polypeptide.

In one embodiment of the present methods, an automated IHC method is used to assay the expression of PD-L1 and/or STK11 on the surface of cells in FFPE tissue specimens. This disclosure provides methods for detecting the presence of human PD-L1 and/or STK11 antigen in a test tissue sample, or quantifying the level of human PD-L1 and/or STK11 antigen or the proportion of cells in the sample that express the antigen, which methods comprise contacting the test sample, and a negative control sample, with a monoclonal antibody that specifically binds to human PD-L1 and/or STK11, under conditions that allow for formation of a complex between the antibody or portion thereof and human PD-L1 and/or STK11. In certain embodiments, the test and control tissue samples are FFPE samples. The formation of a complex is then detected, wherein a difference in complex formation between the test sample and the negative control sample is indicative of the presence of human PD-L1 and/or STK11 antigen in the sample. Various methods are used to quantify PD-L1 and/or STK11 expression.

In a particular embodiment, the automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary antibody; incubating with a postprimary blocking agent; incubating with NovoLink Polymer; adding a chromogen substrate and developing; and counterstaining with hematoxylin.

For assessing PD-L1 and/or STK11 expression in tumor tissue samples, a pathologist examines the number of membrane PD-L1⁺ and/or STK11⁺ tumor cells in each field under a microscope and mentally estimates the percentage of cells that are positive, then averages them to come to the final percentage. The different staining intensities are defined as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%. In one embodiment, the threshold number of cells that needs to be PD-L1 and/or STK11 positive is at least about 100, at least about 125, at least about 150, at least about 175, or at least about 200 cells. In certain embodiments, the threshold number of cells that need to be PD-L1 and/or STK11 positive is at least about 100 cells.

Staining is also assessed in tumor-infiltrating inflammatory cells such as macrophages and lymphocytes. In most cases macrophages serve as an internal positive control since staining is observed in a large proportion of macrophages. While not required to stain with 3+ intensity, an absence of staining of macrophages should be taken into account to rule out any technical failure. Macrophages and lymphocytes are assessed for plasma membrane staining and only recorded for all samples as being positive or negative for each cell category. Staining is also characterized according to an outside/inside tumor immune cell designation. “Inside” means the immune cell is within the tumor tissue and/or on the boundaries of the tumor region without being physically intercalated among the tumor cells. “Outside” means that there is no physical association with the tumor, the immune cells being found in the periphery associated with connective or any associated adjacent tissue.

In certain embodiments of these scoring methods, the samples are scored by two pathologists operating independently, and the scores are subsequently consolidated. In certain other embodiments, the identification of positive and negative cells is scored using appropriate software.

A histoscore (also described as H-score) is used as a more quantitative measure of the IHC data. The histoscore is calculated as follows:

Histoscore=[(% tumor×1 (low intensity))+(% tumor×2 (medium intensity))+(% tumor×3 (high intensity)]

To determine the histoscore, the pathologist estimates the percentage of stained cells in each intensity category within a specimen. Because expression of most biomarkers is heterogeneous the histoscore is a truer representation of the overall expression. The final histoscore range is 0 (no expression) to 300 (maximum expression).

An alternative means of quantifying PD-L1 and/or STK11 expression in a test tissue sample IHC is to determine the adjusted inflammation score (AIS) score defined as the density of inflammation multiplied by the percent PD-L1 and/or STK11 expression by tumor-infiltrating inflammatory cells (Taube et al., “Colocalization of inflammatory response with B7-hl expression in human melanocytic lesions supports an adaptive resistance mechanism of immune escape,” Sci. Transl. Med. 4(127):127ra37 (2012)).

The present methods can treat a tumor at any stage. In certain embodiments, the tumor is derived from an NSCLC of any stage. There are at least seven stages used for NSCLC: occult (hidden) stage, Stage 0 (carcinoma in situ), Stage I, Stage II, Stage IIIA, Stage IIIB, and Stage IV. In the occult stage, the cancer cannot be seen by imaging or bronchoscopy. In Stage 0, cancer cells are found in the lining of the airways.

In one embodiment, the present methods treat a Stage I non-squamous NSCLC. Stage I NSCLC is divided in Stage IA and IB. In Stage IA, the tumor is in the lung only and is 3 centimeters or smaller. In Stage IB, the cancer has not spread to the lymph nodes and one or more of the following is true: 1) the tumor is larger than 3 centimeters but not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus.

In another embodiment, the methods of the present invention treat a Stage II non-squamous NSCLC. Stage II NSCLC is divided into Stage IIA and IIB. In Stage IIA, the cancer has either spread to the lymph nodes or not. If the cancer has spread to the lymph nodes, then the cancer can only have spread to the lymph nodes on the same side of the chest as the tumor, the lymph nodes with cancer or within the lung or near the bronchus. and one or more of the following is true: 1) the tumor is not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIA if the cancer has not spread to the lymph nodes and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. In stage IIB, the cancer has either spread to the lymph nodes or not. If the cancer has spread to the lymph nodes, then the cancer can only have spread to the lymph nodes on the same side of the chest as the tumor, the lymph nodes with cancer are within the lung or near the bronchus and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIB if the cancer has not spread to the lymph nodes and one or more of the following is true: 1) the tumor is larger than 7 centimeters; 2) the cancer has spread to the main bronchus (and is at least 2 centimeters below where the trachea joins the bronchus), the chest wall, the diaphragm, or the nerve that controls the diaphragm; 3) cancer has spread to the membrane around the heart or lining the chest wall; 4) the whole lung has collapsed or developed pneumonitis (inflammation of the lung); or 5) there are one or more separate tumors in the same lobe of the lung.

In other embodiments, any methods of the present invention treat Stage III non-squamous NSCLC. Stage IIIA is divided into 3 sections. These 3 sections are based on 1) the size of the tumor; 2) where the tumor is found and 3) which (if any) lymph nodes have cancer. In the first type of Stage IIIA NSCLC, the cancer has spread to the lymph nodes on the same side of the chest as the tumor, and the lymph nodes with the cancer are near the sternum or where the bronchus enters the lung. Additionally: 1) the tumor may be any size; 2) part of the lung (where the trachea joins the bronchus) or the whole lung may have collapsed or developed pneumonitis (inflammation of the lung); 3) there may be one or more separate tumors in the same lobe of the lung; and 4) cancer can have spread to any of the following: a) main bronchus, but not the area where the trachea joins the bronchus, b) chest well, c) diaphragm and the nerve that controls it, d) membrane around the lung or lining the chest wall, e) membrane around the heart. In the second type of Stage IIIA NSCLC, the cancer has spread to the lymph nodes on the same side of the chest as the tumor, and the lymph nodes with the cancer are within the lung or near the bronchus. Additionally: 1) the tumor may be any size; 2) the whole lung may have collapsed or developed pneumonitis (inflammation of the lung); 3) there may be one or more separate tumors in the any of the lobes of the lung with cancer; and 4) cancer can have spread to any of the following: a) main bronchus, but not the area where the trachea joins the bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the third type of Stage IIIA NSCLC, the cancer has not spread to the lymph nodes, the tumor may be any size, and cancer has spread to any one of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi). Stage IIIB is divided into 2 sections depending on 1) the size of the tumor, 2) where the tumor is found, and 3) which lymph nodes have cancer. In the first type of Stage IIIB NSCLC, the cancer has spread to the lymph nodes on the opposite side of the chest as the tumor. Additionally, 1) the tumor may be any size; 2) part of the lung (where the trachea joins the bronchus) or the whole lung may have collapsed or developed pneumonitis (inflammation of the lung); 3) there may be one or more separate tumors in any of the lobs of the lung with cancer; and 4) cancer may have spread to any of the following: a) main bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the second type of Stage IIIB NSCLC, the cancer has spread to lymph nodes on the same side of the chest as the tumor. The lymph nodes with cancer are near the sternum (chest bone) or where the bronchus enters the lung. Additionally, 1) the tumor may be any size; 2) there may be separate tumors in different lobes of the same lung; and 3) cancer has spread to any of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi).

In some embodiments, the methods of the invention treat a Stage IV non-squamous NSCLC. In Stage IV NSCLC, the tumor may be any size and the cancer may have spread to the lymph nodes. One or more of the following is true in Stage IV NSCLC: 1) there are one or more tumors in both lungs; 2) cancer is found in the fluid around the lungs or heart; and 3) cancer has spread to other parts of the body, such as the brain, liver, adrenal glands, kidneys or bone.

In certain embodiments of the present methods, the anti-PD-1 antibody is nivolumab. In other embodiments, it is pembrolizumab. Typically, the anti-PD-1 antibodies are formulated for intravenous administration. In certain embodiments, the anti-PD-1 antibody is administered by intravenous infusion over a period of 60 minutes. In certain embodiments, the anti-PD-1 antibody is administered as a pharmaceutically acceptable formulation. In certain embodiments, the anti-PD-1 antibody or antigen-binding portion thereof is administered at a subtherapeutic dose.

### Anti-PD-1 Antibodies or Anti-PD-L1 Antibodies Useful for the Invention

Human monoclonal antibodies that bind specifically to PD-1 with high affinity have been disclosed in U.S. Pat. No. 8,008,449. Other anti-PD-1 monoclonal antibodies have been described in, for example, U.S. Pat. Nos. 6,808,710, 7,488,802, 8,168,757 and 8,354,509, and PCT Publication No. WO 2012/145493. Each of the anti-PD-1 human monoclonal antibodies disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and (j) inhibits tumor cell growth in vivo. Anti-PD-1 antibodies usable in the present invention include monoclonal antibodies that bind specifically to human PD-1 and exhibit at least one, in some embodiments, at least five, of the preceding characteristics. In some embodiments, the anti-PD-1 antibody is nivolumab. In one embodiment, the anti-PD-1 antibody is pembrolizumab.

In one embodiment, the anti-PD-1 antibody is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of anti-tumor T-cell functions (U.S. Pat. No. 8,008,449; Wang et al., In vitro characterization of the anti-PD-1 antibody nivolumab, BMS-936558, and in vivo toxicology in non-human primates, Cancer Imm Res, 2(9):846-56 (2014)). In another embodiment, the anti-PD-1 antibody or fragment thereof cross-competes with nivolumab. In other embodiments, the anti-PD-1 antibody or fragment thereof binds to the same epitope as nivolumab. In certain embodiments, the anti-PD-1 antibody has the same CDRs as nivolumab.

In another embodiment, the anti-PD-1 antibody (or antigen-binding portion thereof) cross-competes with pembrolizumab. In some embodiments, the anti-PD-1 antibody binds to the same epitope as pembrolizumab. In certain embodiments, the anti-PD-1 antibody has the same CDRs as pembrolizumab. In another embodiment, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also www.cancer.gov/drugdictionary?cdrid=695789 (last accessed: Dec. 14, 2014). Pembrolizumab has been approved by the FDA for the treatment of relapsed or refractory melanoma.

In other embodiments, the anti-PD-1 antibody (or antigen-binding portion thereof) cross-competes with MEDI0680. In still other embodiments, the anti-PD-1 antibody or fragment thereof binds to the same epitope as MEDI0680. In certain embodiments, the anti-PD-1 antibody has the same CDRs as MEDI0680. In other embodiments, the anti-PD-1 antibody is MEDI0680 (formerly AMP-514), which is a monoclonal antibody. MEDI0680 is described, for example, in U.S. Pat. No. 8,609,089B2.

In certain embodiments, an immune checkpoint inhibitor is AMP-224, which is a B7-DC Fc fusion protein. AMP-224 is discussed in U.S. Publ. No. 2013/0017199 or in www.cancer.gov/publications/dictionaries/cancer-drug?cdrid=700595 (last accessed Jul. 8, 2015).

In other embodiments, the anti-PD-1 antibody (or antigen-binding portion thereof) cross-competes with BGB-A317. In some embodiments, the anti-PD-1 antibody binds the same epitope as BGB-A317. In certain embodiments, the anti-PD-1 antibody has the same CDRs as BGB-A317. In certain embodiments, the anti-PD-1 antibody is BGB-A317, which is a humanized monoclonal antibody. BGB-A317 is described in U.S. Publ. No. 2015/0079109.

In other embodiments, the anti-PD-1 antibody (or antigen-binding portion thereof) cross-competes with INCSHR1210 (SHR-1210). In some embodiments, the anti-PD-1 antibody binds to the same epitope as INCSHR1210 (SHR-1210). In certain embodiments, the anti-PD-1 antibody has the same CDRs as INCSHR1210 (SHR-1210). In certain embodiments, the anti-PD-1 antibody is INCSHR1210 (SHR-1210), which is a human monoclonal antibody. INCSHR1210 (SHR-1210) is described in WO2015/085847.

In other embodiments, the anti-PD-1 antibody (or antigen-binding portion thereof) cross-competes with REGN-2810. In some embodiments, the anti-PD-1 antibody binds to the same epitope as REGN-2810. In certain embodiments, the anti-PD-1 antibody has the same CDRs as REGN-2810. In certain embodiments, the anti-PD-1 antibody is REGN-2810, which is a human monoclonal antibody. REGN-2810 is described in WO2015/112800.

In other embodiments, the anti-PD-1 antibody (or antigen-binding portion thereof) cross-competes with PDR001. In some embodiments, the anti-PD-1 antibody binds to the same epitope as PDR001. In certain embodiments, the anti-PD-1 antibody has the same CDRs as PDR001. In certain embodiments, the anti-PD-1 antibody is PDR001, which is a humanized monoclonal antibody. PDR001 is described in WO2015/112900.

In other embodiments, the anti-PD-1 antibody (or antigen-binding portion thereof) cross-competes with TSR-042 (ANB011). In some embodiments, the anti-PD-1 antibody binds to the same epitope as TSR-042 (ANB011). In certain embodiments, the anti-PD-1 antibody has the same CDRs as TSR-042 (ANB011). In certain embodiments, the anti-PD-1 antibody is TSR-042 (ANB011), which is a humanized monoclonal antibody. TSR-042 (ANB011) is described in WO2014/179664.

In other embodiments, the anti-PD-1 antibody (or antigen-binding portion thereof) cross-competes with STI-1110. In some embodiments, the anti-PD-1 antibody binds to the same epitope as STI-1110. In certain embodiments, the anti-PD-1 antibody has the same CDRs as STI-1110. In certain embodiments, the anti-PD-1 antibody is STI-1110, which is a human monoclonal antibody. STI-1110 is described in W2014/194302.

Anti-PD-1 antibodies usable in the disclosed methods also include isolated antibodies that bind specifically to human PD-1 and cross-compete for binding to human PD-1 with nivolumab (see, e.g., U.S. Pat. Nos. 8,008,449 and 8,779,105; WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 antibody, nivolumab are monoclonal antibodies. For administration to human subjects, these cross-competing antibodies are chimeric antibodies, humanized antibodies, or human antibodies. Such chimeric, humanized, or human monoclonal antibodies can be prepared and isolated by methods well known in the art.

Anti-PD-1 antibodies usable in the methods of the disclosed invention also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H}I domains; (iv) a Fv fragment consisting of the V_{L} and V_{H} domains of a single arm of an antibody; or any combination thereof.

Anti-PD-1 antibodies suitable for use in the disclosed methods or compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain embodiments, the antibody is a humanized antibody. In other embodiments, the antibody is a human antibody. antibodies of an IgG1, IgG2, IgG3, or IgG4 isotype can be used.

In certain embodiments, the anti-PD-1 antibody or antigen-binding portion thereof comprises a heavy chain constant region that is of a human IgG1 or IgG4 isotype. In certain other embodiments, the sequence of the IgG4 heavy chain constant region of the anti-PD-1 antibody or antigen-binding portion thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014 Cancer Immunol Res. 2(9):846-56). In yet other embodiments, the antibody comprises a light chain constant region that is a human kappa or lambda constant region. In other embodiments, the anti-PD-1 antibody or antigen-binding portion thereof is a monoclonal antibody or an antigen-binding portion thereof. In certain embodiments of any of the therapeutic methods described herein comprising administration of an anti-PD-1 antibody, the anti-PD-1 antibody is nivolumab. In other embodiments, the anti-PD-1 antibody is pembrolizumab. In other embodiments, the anti-PD-1 antibody is chosen from the human antibodies 17D8, 2D3, 4H1, 4A11, 7D3, and 5F4 described in U.S. Pat. No. 8,008,449. In still other embodiments, the anti-PD-1 antibody is MEDI0680 (formerly AMP-514), AMP-224, or BGB-A317.

In certain embodiments, an anti-PD-1 antibody used in the methods can be replaced with another anti-PD-1 or anti-PD-L1 antagonist. For example, because an anti-PD-L1 antibody prevents interaction between PD-1 and PD-L1, thereby exerting similar effects to the signaling pathway of PD-1, an anti-PD-L1 antibody can replace the use of an anti-PD-1 antibody in the methods disclosed herein. Therefore, in one embodiment, the present invention is directed to a method for treating a subject afflicted with a tumor comprising administering to the subject a therapeutically effective amount an anti-PD-L1 antibody. In certain embodiments, the anti-PD-L1 antibody useful for the method is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446 or atezolizumab) (see, e.g., Herbst et al., (2013) J Clin Oncol 31(suppl):3000. Abstract; U.S. Pat. No. 8,217,149), MEDI4736 (also called Durvalumab; Khleif (2013) In: Proceedings from the European Cancer Congress 2013; Sep. 27-Oct. 1, 2013; Amsterdam, The Netherlands. In other embodiments, the anti-PD-L1 antibody is CX-072 (also called CytomX; See WO2016/149201). In other embodiments, the anti-PD-L1 monoclonal antibody is selected from the group consisting of 28-8, 28-1, 28-12, 29-8, 5H1, and any combination thereof. In certain embodiments, the antibodies that cross-compete for binding to human PD-L1 with, or bind to the same epitope region of human PD-L1 as the above-references PD-L1 antibodies are monoclonal antibodies. For administration to human subjects, these cross-competing antibodies can be chimeric antibodies, humanized antibodies, or human antibodies. Such chimeric, humanized, or human monoclonal antibodies can be prepared and isolated by methods well known in the art. Abstract 802, See U.S. Pat. No. 8,779,108 or US 2014/0356353, filed May 6, 2014), or MSB0010718C (also called Avelumab; See US 2014/0341917).

### Standard-of-Care Therapies for Lung Cancer

Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES® (2014), available at: www.nccn.org/professionals/physician_gls/f_guidelines.asp, last accessed May 14, 2014).

NSCLC is the leading cause of cancer death in the U.S. and worldwide, exceeding breast, colon and prostate cancer combined. In the U.S., an estimated 228,190 new cases of lung and bronchial will be diagnosed in the U.S., and some 159,480 deaths will occur because of the disease (Siegel et al. (2014) CA Cancer J Clin 64(1):9-29). The majority of patients (approximately 78%) are diagnosed with advanced/recurrent or metastatic disease. Metastases to the adrenal gland from lung cancer are a common occurrence, with about 33% of patients having such metastases. NSCLC therapies have incrementally improved OS, but benefit has reached a plateau (median OS for late stage patients is just 1 year). Progression after 1L therapy occurred in nearly all of these subjects and the 5-year survival rate is only 3.6% in the refractory setting. From 2005 to 2009, the overall 5-year relative survival rate for lung cancer in the U.S. was 15.9% (NCCN GUIDELINES®, Version 3.2014—Non-Small Cell Lung Cancer, available at: www.nccn.org/professionals/physician_gls/pdf/nscl.pdf, last accessed May 14, 2014).

Surgery, radiation therapy (RT), and chemotherapy are the three modalities commonly used to treat NSCLC patients. As a class, NSCLCs are relatively insensitive to chemotherapy and RT, compared to small cell carcinoma. In general, for patients with Stage I or II disease, surgical resection provides the best chance for cure, with chemotherapy increasingly being used both pre-operatively and post-operatively. RT can also be used as adjuvant therapy for patients with resectable NSCLC, the primary local treatment, or as palliative therapy for patients with incurable NSCLC.

Patients with Stage IV disease who have a good performance status (PS) benefit from chemotherapy. Many drugs, including platinum agents (e.g., cisplatin, carboplatin), taxanes agents (e.g., paclitaxel, albumin-bound paclitaxel, docetaxel), vinorelbine, vinblastine, etoposide, pemetrexed and gemcitabine are useful for Stage IV NSCLC. Combinations using many of these drugs produce 1-year survival rates of 30% to 40% and are superior to single agents. Specific targeted therapies have also been developed for the treatment of advanced lung cancer. For example, bevacizumab (AVASTIN) is a monoclonal antibody that blocks vascular endothelial growth factor A (VEGF-A). Erlotinib (TARCEVA®) is a small-molecule TKI of epidermal growth factor receptor (EGFR). Crizotinib (XALKORI) is a small-molecule TKI that targets ALK and MET, and is used to treat NSCLC in patients carrying the mutated ALK fusion gene. Cetuximab (ERBITUX®) is a monoclonal antibody that targets EGFR.

There is a particular unmet need among patients who have squamous cell NSCLC (representing up to 25% of all NSCLC) as there are few treatment options after first line (1L) therapy. Single-agent chemotherapy is standard of care following progression with platinum-based doublet chemotherapy (Pt-doublet), resulting in median OS of approximately 7 months. Docetaxel remains the benchmark treatment in this line of therapy although erlotinib can also be used with less frequency. Pemetrexed has also been shown to produce clinically equivalent efficacy outcomes but with significantly fewer side effects compared with docetaxel in the second line (2L) treatment of patients with advanced NSCLC (Hanna et al. (2004) J Clin Oncol 22:1589-97). No therapy is currently approved for use in lung cancer beyond the third line (3L) setting. Pemetrexed and bevacizumab are not approved in squamous NSCLC, and molecularly targeted therapies have limited application. The unmet need in advanced lung cancer has been compounded by the recent failure of Oncothyreon and Merck KgaA's STIMUVAX® to improve OS in a phase 3 trial, inability of ArQule's and Daiichi Sankyo's c-Met kinase inhibitor, tivantinib, to meet survival endpoints, failure of Eli Lilly's ALIMTA® in combination with Roche's AVASTIN® to improve OS in a late-stage study, and Amgen's and Takeda Pharmaceutical's failure to meet clinical endpoints with the small-molecule VEGF-R antagonist, motesanib, in late-stage trials.

### Immunotherapy of Lung Cancer

A clear need exists for effective agents for patients who have progressed on multiple lines of targeted therapy, as well as for therapies that extend survival for longer periods beyond the current standard treatments. Newer approaches involving immunotherapy, especially blockade of immune checkpoints including the CTLA-4, PD-1, and PD-L1 inhibitory pathways, have recently shown promise (Creelan et al. (2014) Cancer Control 21(1):80-89). However, a need remains to identify patients that may be more responsive to immunotherapy, in particular to identify patients that are more likely to respond to an anti-PD-1 or anti-PD-L1 antibody therapy.

### Pharmaceutical Compositions and Dosages

Therapeutic agents of the present invention can be constituted in a composition, e.g., a pharmaceutical composition containing one or more antibodies and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. In one embodiment, the carrier for a composition containing an antibody is suitable for intravenous, intramuscular, subcutaneous, parenteral, spinal, or epidermal administration (e.g., by injection or infusion). A pharmaceutical composition of the invention can include one or more pharmaceutically acceptable salts, anti-oxidant, aqueous and non-aqueous carriers, and/or adjuvants such as preservatives, wetting agents, emulsifying agents, and dispersing agents.

The present disclosure provides dosage regimens that can provide a desired response, e.g., a maximal therapeutic response and/or minimal adverse effects. For administration of an anti-PD-1 antibody, the dosage can range from about 0.01 to about 10 mg/kg, about 1 to about 9 mg/kg, about 2 to about 8 mg/kg, about 3 to about 7 mg/kg, about 3 to about 6 mg/kg, 0.01 to about 5 mg/kg, or about 1 to about 3 mg/kg of the subject's body weight. For example, dosages can be about 0.1, about 0.3, about 1, about 2, about 3, about 4, about 5, about 6, about 7, about 8, about 9, or about 10 mg/kg body weight. The dosing schedule is typically designed to achieve exposures that result in sustained receptor occupancy (RO) based on typical pharmacokinetic properties of an antibody. An exemplary treatment regime entails administration once about per week, once about every 2 weeks, once about every 3 weeks, once about every 4 weeks, once about every month, once about every 3-6 months or longer. In certain embodiments, an anti-PD-1 antibody such as nivolumab is administered to the subject once about every 2 weeks. The anti-PD-1 antibody can be administered in at least two doses, each of the doses is at an amount of about 0.01 mg/kg to about 5 mg/kg, e.g., 3 mg/kg, at a dosing interval of every two weeks between the two doses. In some embodiments, the anti-PD-1 antibody is administered in at least three, four, five, six, or seven doses (i.e., multiple doses), each of the doses is at an amount of about 0.01 mg/kg to about 10 mg/kg, e.g., 1 mg/kg, 3 mg/kg, or 6 mg/kg, at a dosing interval of every two weeks between two adjacently given doses. The dosage and scheduling can change during a course of treatment. In one embodiment, a dosage regimen for an anti-PD-1 antibody of the invention comprises about 0.1 to about 5 mg/kg body weight, about 1 to about 5 mg/kg body weight, or about 1 to about 3 mg/kg body weight via intravenous administration, with the antibody being given every about 14-21 days in up to about 6-week or about 12-week cycles until complete response or confirmed progressive disease. In some embodiments, the antibody treatment, or any combination treatment disclosed herein, is continued for at least about 1 month, at least about 3 months, at least about 6 months, at least about 9 months, at least about 1 year, at least about 18 months, at least about 24 months, at least about 3 years, at least about 5 years, or at least about 10 years.

When used in combinations with other therapies (e.g., other immunotherapies), the dosage of an anti-PD-1 antibody can be lowered compared to the monotherapy dose. Dosages of nivolumab that are lower than the typical 3 mg/kg, but not less than 0.001 mg/kg, are subtherapeutic dosages. The subtherapeutic doses of an anti-PD-1 antibody used in the methods herein are higher than 0.001 mg/kg and lower than 3 mg/kg. In some embodiments, a subtherapeutic dose is about 0.001 mg/kg-about 1 mg/kg, about 0.01 mg/kg to about 1 mg/kg, about 0.1 mg/kg to about 1 mg/kg, or about 0.001 mg/kg to about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.5 mg/kg, or at least about 1.0 mg/kg body weight. Receptor-occupancy data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%) (Brahmer et al. (2010) J Clin Oncol 28:3167-75). Thus, 0.3 mg/kg dosing can allow for sufficient exposure to lead to maximal biologic activity.

In some embodiments of the invention, the anti-PD-1 antibody is administered at a dose of 3 mg/kg. In other embodiments of the invention, the anti-PD-1 antibody is administered at a dose of 1 mg/kg.

In certain embodiments, the dose of an anti-PD-1 antibody (or an anti-PD-L1 antibody) is a fixed dose in a pharmaceutical composition. In other embodiments, the method of the present invention can be used with a flat dose (a dose given to a patient irrespective of the body weight of the patient). In embodiments, the flat dose of the anti-PD-1 antibody or antigen binding portion thereof is at least about 100 mg, 120 mg, 140 mg, 160 mg, 180 mg, 200 mg, 220 mg, 240 mg, 260 mg, 280 mg, 300 mg, 400 mg, 420 mg, 440 mg, 460 mg, 480 mg, 500 mg, 520 mg, 540 mg, 560 mg, or 600 mg. For example, a flat dose of a nivolumab can be about 240 mg. For example, a flat dose of pembrolizumab can be about 200 mg. In embodiments, the anti-PD-1 antibody or antigen-binding portion thereof is administered at a dose of about 240 mg. In embodiments, the anti-PD-1 antibody or antigen-binding portion thereof is administered at a dose of about 360 mg. In embodiments, the anti-PD-1 antibody or antigen-binding portion thereof is administered at a dose of about 480 mg. In embodiments, the flat dose of the anti-PD-1 antibody or antigen binding portion thereof is administered once about every week, every two weeks, every three weeks, every four weeks, every five weeks, or every six weeks. In one embodiment, 360 mg of the anti-PD-1 antibody or antigen binding fragment is administered once every 3 weeks. In another embodiment, 480 mg of the anti-PD-1 antibody or antigen binding fragment is administered once every 4 weeks.

Dosage and frequency vary depending on the half-life of the antibody in the subject. In general, human antibodies show the longest half-life, followed by humanized antibodies, chimeric antibodies, and non-human antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, or until the patient shows partial or complete amelioration of symptoms of disease. Thereafter, the patient can be administered a prophylactic regime.

Actual dosage levels of the active ingredients in the pharmaceutical compositions of the present invention can be varied so as to obtain an amount of the active ingredient that is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds, and/or materials used in combination with the particular compositions employed, the age, sex, weight, condition, general health, and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results.

### Kits

Also within the scope of the present invention are kits comprising an anti-PD-1 antibody or an anti-PD-L1 antibody. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a tumor, the kit comprising: (a) a dosage ranging from about 4 mg to about 500 mg of an anti-PD-1 antibody; and (b) instructions for using the anti-PD-1 antibody in any method described herein. In certain embodiments for treating human patients, the kit comprises an anti-human PD-1 antibody disclosed herein, e.g., nivolumab or pembrolizumab. In some embodiments, the kit further comprises an anti-PD-L1 antibody and/or an anti-STK11 antibody. In other embodiments, the kit further comprises instructions for detecting the expression of PD-L1 and/or STK11 in a tumor sample.

The present invention is further illustrated by the following example that should not be construed as further limiting. The contents of all references cited throughout this application are expressly incorporated herein by reference.

### Example 1

### STK11 Mutation as a Biomarker for Nivolumab Response

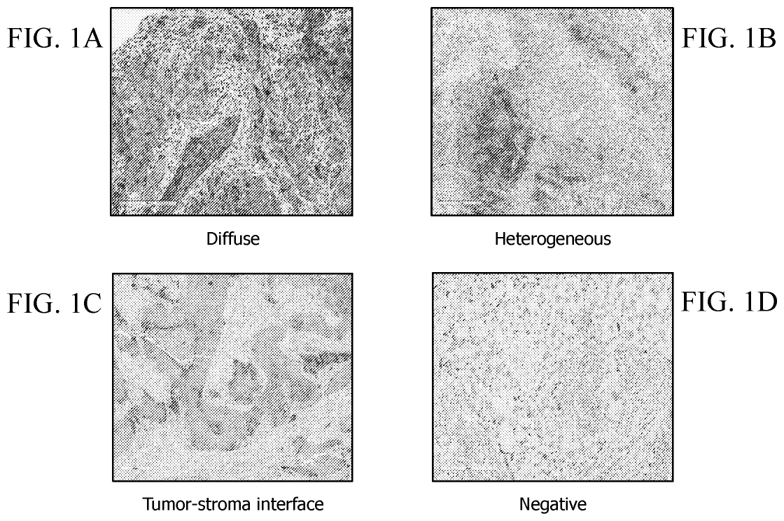

PD-L1 is expressed in NSCLC tumors, e.g., commercial NSCLC tumors, according to different patterns of expressing (FIGS. 1A-1D). These patterns were designated as diffuse, heterogenous, tumor-stroma interface, and negative. PD-L1 expression patterns can be linked to mechanistic hypotheses. For example, in tumors with diffuse patterns, the expression of PD-L1 is driven by 9p24 amplification in oncogenic signaling pathway instead of being driven by mutation. In tumors with tumor-stroma interface patterns, there are adaptive resistances instead of epithelial to mesenchymal transition (EMT).

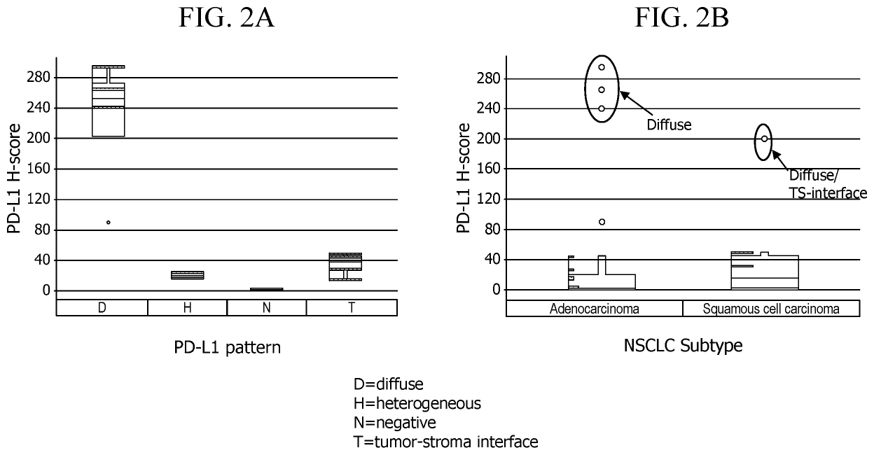

Patterns of PD-L1 expression in NSCLC commercial tumors correlate with PD-L1 H-score, as shown in FIGS. 2A-2B. There is a substantial difference in the level of PD-L1 expression according to each pattern. For example, very high H-scores were observed in diffuse pattern samples, which suggested a potential dependence of these tumors on PD-L1 inhibition.

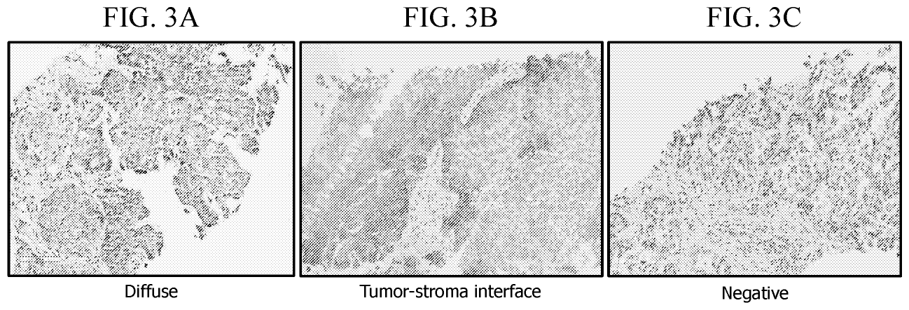

The PD-L1 expression patterns observed in NSCLC commercial tumors were also observed in biopsies. FIGS. 3A-3C shows PD-L1 expression patterns in trial biopsies corresponding to patients treated with nivolumab monotherapy, which corresponding to the same patterns observed in NSCLC commercial tumors.

The potential for false negative PD-L1 is impacted by pattern category, preanalytic variables, and by the size of the biopsy. For example, tumor-stroma interface patterns are heterogeneous and particularly susceptible to false negative results. Accordingly, there is a need for biomarkers that can facilitate the classification of NSCLC tumors, which in turn could be used to predict the tumor response to a certain therapeutic agent.

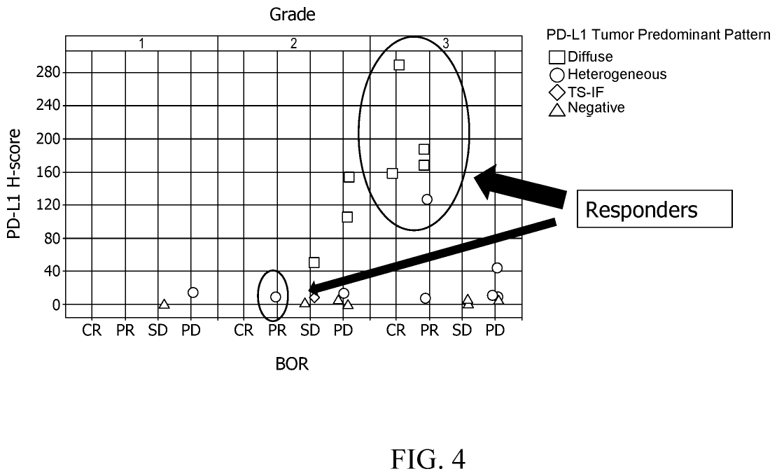

A correlation between patterns of PD-L1 expression and nivolumab efficacy was observed (FIG. 4). Most of complete responders in Grade 3 tumors had a diffuse pattern of PD-L1 expression, and high PD-L1 H-score. Accordingly, the identification of biomarkers specific for the diffuse PD-L1 expression pattern could be used to identify patients suitable for treatment for nivolumab based on the presence/absence of that biomarker.

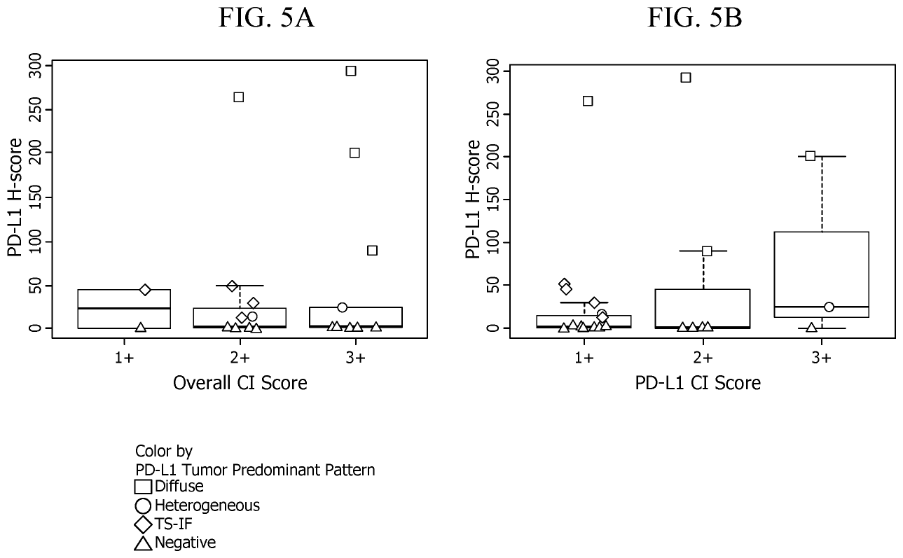

The immune infiltrate can be potentially used as biomarker specific for NSCLC tumors with diffuse expression patterns (FIGS. 5A-5B), since in the commercial NSCLC tumors used to generate the data presented in FIG. 5, those with diffuse or heterogenous PD-L1 patterns were associated with more abundant immune infiltrate (higher PD-L1 H-score).

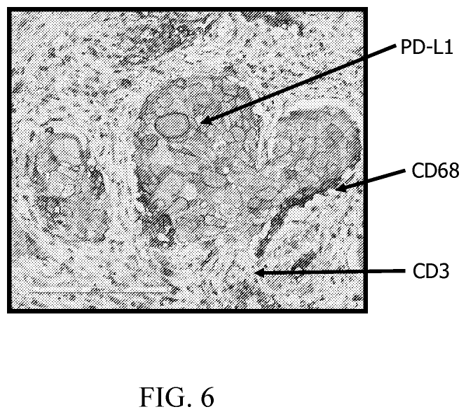

Multiplex IHC experiments showed defined special relationships between the tumor cells and immune cell subsets. PD-L1 labeling showed diffuse PD-L1 expression in the tumor. CD68 detection indicated that a layer of macrophages at the tumor-stroma interface contribute to the formation of a “barrier” activated T-cells, whereas CD3 detection indicated that T-cells are moderately abundant but largely confined to the stroma (FIG. 6).

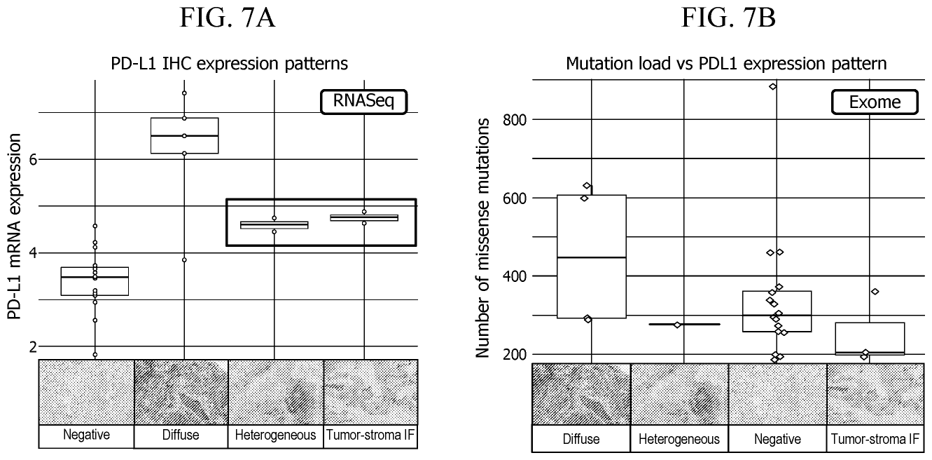

PD-L1 expression patterns correlate with genomic data (FIGS. 7A-7B). FIG. 7 A shows that the level of PD-L1 expression correlates with RNA sequencing data, but RNA sequencing data alone does not provide geographical context for the PD-L1 expression patterns observed via IHC. FIG. 7B which presents exome sequencing data shows that a PD-L1 diffuse expression pattern correlates with a higher mutation load.

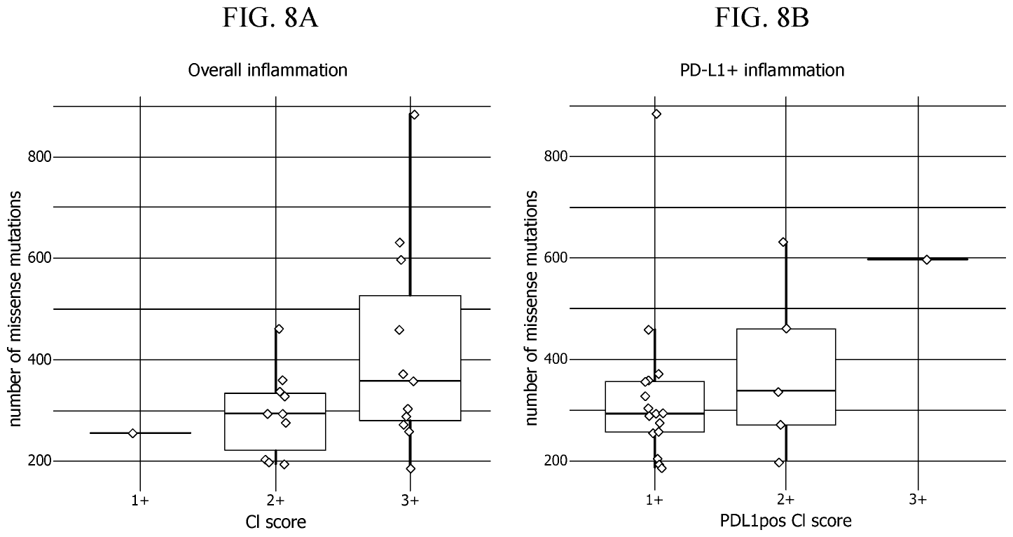

A higher mutational load has also been linked to inflamed tumors (FIGS. 8A-8B). FIG. 8A shows overall inflammation measured using a “CI Score” which is the intensity score of chronic inflammation infiltrate. FIG. 8B shows PD-L1+ inflammation measured using a “PD-L1+ CI Score” which is the intensity score of relative proportion of PD-L1+ immune infiltrate. There is a relationship between the number of missense mutations in NSCLC tumors and overall inflammation.

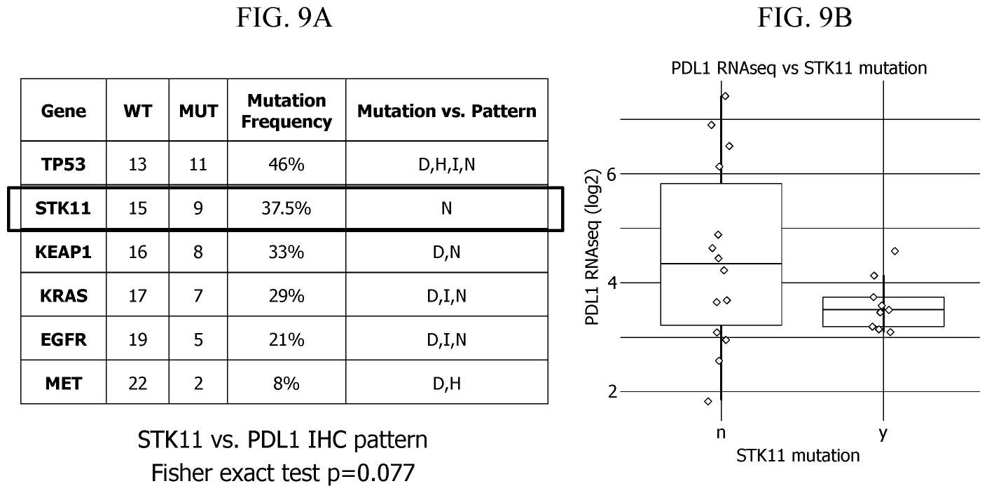

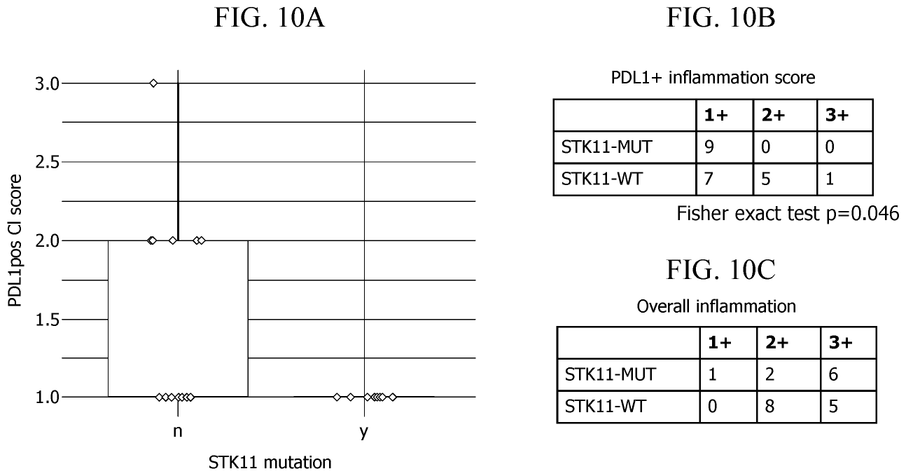

Frequency of mutations in different biomarkers (TP53, STK11, KEAP1, KRAS, EGFR, and MET) versus the observed PD-L1 expression pattern was evaluated (FIGS. 9A-9B), the results indicating that negative PD-L1 tumor cell expression and lower PD-L1 mRNA expression are been associated with the presence of SKT11 mutation. The presence of mutant STK11 correlated with the presence of the “N” (PD-L1 negative) expression pattern (FIG. 10A) and inflammation (FIGS. 10B-10C). The presence of mutant STK11 did not correlate with the presence of the “D” (diffuse) pattern, which is the pattern observed in most responders to nivolumab therapy. Accordingly, the presence of mutant forms of STK11 could be used as negative biomarker for treatment of NSCLC tumors with nivolumab (i.e., its presence would predict lack of response or poor response to nivolumab). Conversely, the presence of wild type forms of STK11 (or absence of mutant forms) could be used as a positive selection biomarker for treatment with nivolumab.

STK11 loss due to mutation is predicted to increase mTOR signaling. Lung adenocarcinoma (both mouse model and human tumors) with KRAS and STK11 mutations show decreased expression of PD-L1 and diminished T-cell infiltrates. The proposed mechanism of immune suppression mediated by mutations in SKT11 would include a switch to glycolytic metabolism with increased lactate production, and a frequent co-mutation of KEP1 leading to an anti-inflammatory transcriptional program.

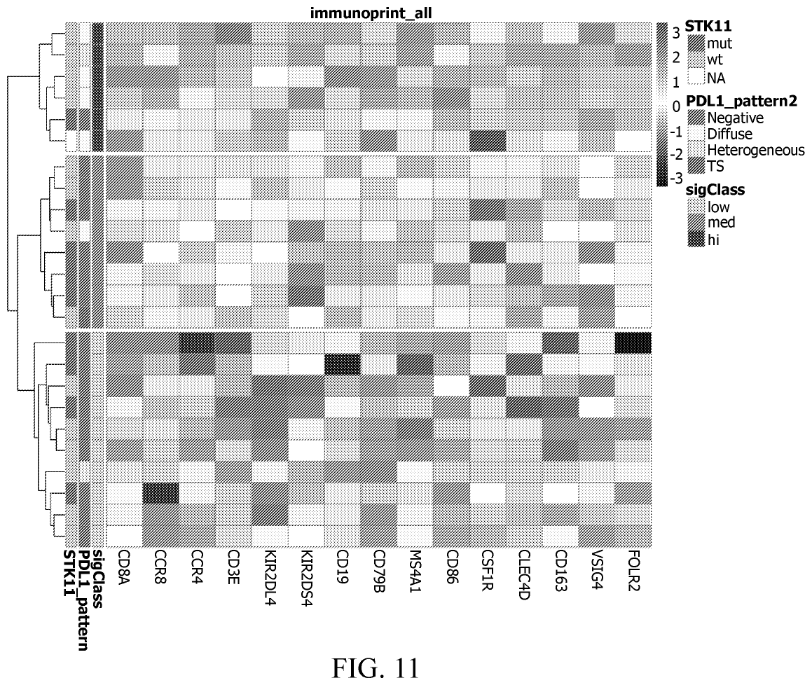

An immunoprint analysis of 24 NSCLC tumor samples in which levels of FOLR2, VSIG4, CD163, CLEC4D, CSF1R, CD86, MS4A1, CD79B, CD19, KIR2DS4, CD3E, CCR4, CCR8, and CD8A were analyzed was used to classify the samples according to inflammation patterns (sigClass). See FIG. 11. Samples were classified as low (“sigClass low”), medium (“sigClass med”), and high (“sigClass hi”) inflammation. Samples were classified also according to presence (“STK11 mut”) or absence (“STK11 wt”) of STK11 mutations. In addition, samples were classified according to PD-L1 expression pattern as negative (“PDL1_pattern 2 Negative”), diffuse, (“PDL1_pattern 2 Diffuse”), heterogenous (“PDL1_pattern 2 Heterogeneous”), and tumor-stroma interface (“PDL1_pattern 2 TS”). Tumor with diffuse PD-L1 expression patterns were shown highly inflamed and presented the wild type form of STK11. PD-L1 negative tumors cluster in two groups: moderate inflammation and low inflammation. There was no clear distinction in level of inflammation of PD-L1 negative tumors by STK11 mutation status. All the tumors with mutant STK11 were also negative for PD-L1.

This data indicate that PD-L1 patterns of expression associate with unique phenotypic and genetic backgrounds. Diffuse PD-L1 expression correlates with an inflamed TME and higher mutational load. Furthermore, the presence of STK11 mutations identifies a subset of PD-L1 negative tumors. These findings establish the suitability of STK11 as a biomarker to identify a subset of PD-L1 negative tumors, and the possibility of integrating histopathologic and genomic data to identify features that define NSCLC subsets with varying likelihood of response to immunotherapy.

What is claimed is:

1. An antibody or an antigen-binding portion thereof that binds specifically to a Programmed Death-1 (PD-1) receptor and inhibits PD-1 activity (“anti-PD-1 antibody”) for use in a method of treating a subject afflicted with a tumor, the method comprising (i) determining an expression pattern of PD-L1 in the tumor, and (ii) administering the anti-PD-1 antibody if the tumor exhibits a diffuse pattern of PD-L1 expression, if the tumor exhibits a tumor stroma interface pattern of PD-L1 expression, and/or if the tumor exhibits a heterogeneous pattern of PD-L1 expression.

2. An anti-PD-1 antibody for use in a method of treating a subject afflicted with a tumor, the method comprising administering to the subject the anti-PD-1 antibody wherein the tumor exhibits a diffuse pattern of PD-L1 expression, if the tumor exhibits a tumor stroma interface pattern of PD-L1 expression, and/or if the tumor exhibits a heterogeneous pattern of PD-L1 expression.

3. The anti-PD-1 antibody for use according to claim 1 or 2, wherein the method further comprises identifying the patient as having a tumor that expresses STK11 (“STK11 positive tumor”) prior to the administration of the anti-PD-1 antibody.

4. An anti-PD-1 antibody for use in a method of treating a subject afflicted with a tumor, the method comprising administering to the subject the anti-PD-1 antibody, wherein the tumor is STK11 positive tumor.

5. The anti-PD-1 antibody for use according to claim 3 or 4, wherein STK11 is wild-type STK11.

6. The anti-PD-1 antibody for use according to any one of claims 1 to 5, wherein the tumor is derived from a lung cancer, optionally a small cell lung cancer (SCLC) or a non-small cell lung cancer (NSCLC).

7. The anti-PD-1 antibody for use according to any one of claims 1 to 6, wherein (i) the diffuse pattern of PD-L1 expression is characterized by a PD-L1 H-score of from about 60 to about 500, from about 80 to about 480, from about 100 to about 460, from about 120 to about 440, from about 140 to about 420, from about 160 to about 400, from about 180 to about 380, from about 200 to about 360, from about 200 to about 340, from about 200 to about 320, or from about 200 to about 300; (ii) the diffuse pattern of PD-L1 expression is characterized by a PD-L1 H-score of at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 225, at least about 250, at least about 275, or at least about 300; (iii) the heterogeneous pattern of PD-L1 expression is characterized by a PD-L1 H-score of from about 1 to about 50, from about 5 to about 45, from about 10 to about 40, or from about 15 to about 35, and wherein the PD-L1 expression is restricted to one or more distinct portions of the tumor; or (iv) the heterogeneous pattern of PD-L1 expression is characterized by a PD-L1 H-score of at least about 5, at least about 10, at least about 15, at least about 20, at least about 25, at least about 30, at least about 35, or at least about 40.

8. The anti-PD-1 antibody for use according to any one of claims 1 to 7, wherein the tumor is characterized by having at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of tumor cells expressing PD-L1.

9. The anti-PD-1 antibody for use according to any one of claims 1 to 8, wherein the STK11-positive tumor is characterized by having at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of tumor cells expressing STK11.

10. The anti-PD-1 antibody for use according to any one of claims 1 to 9, wherein the tumor exhibits high inflammation.

11. The anti-PD-1 antibody for use according to any one of claims 1 to 10, wherein (i) the anti-PD-1 antibody cross-competes with nivolumab for binding to human PD-1, (ii) the anti-PD-1 antibody binds to the same epitope as nivolumab, (iii) the anti-PD-1 antibody is nivolumab, or (iv) the anti-PD-1 antibody is nivolumab.

12. The anti-PD-1 antibody for use according to any one of claims 1 to 11, wherein (i) the anti-PD-1 antibody is administered at a dose ranging from at least about 0.1 mg/kg to at least about 10.0 mg/kg body weight once about every 1, 2, or 3 weeks or (ii) the anti-PD-1 antibody is administered at a dose of at least about 3 mg/kg body weight once about every 2 weeks.

13. The anti-PD-1 antibody for use according to any one of claims 1 to 11, wherein the anti-PD-1 antibody is administered at a flat dose, optionally at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, or at least about 550 mg, about once every 1, 2, 3 or 4 weeks.

14. The anti-PD-1 antibody for use according to any one of claims 1 to 13, wherein (i) the administering reduces the size of the tumor, optionally by at least about 10%, about 20%, about 30%, about 40%, or about 50% compared to the tumor size prior to the administration; (ii) the administering provides progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration; (iii) the administering results in stable disease after the administration; (iv) the administering results in a partial response after the administration; or (v) the administering results in a complete response after the administration.

15. A kit comprising:
(a) a dosage ranging from about 4 mg to about 500 mg of antibody or an antigen-binding portion thereof that binds specifically to a Programmed Death-1 (PD-1) receptor and inhibits PD-1 activity (“an anti-PD-1 antibody”); and
(b) instructions for using the anti-PD-1 antibody according to any one of claims 1 to 14.
